(19) United States
(12) Patent Application Publication
(10) Pub. No.: US 2026/0294815 A1
(43) Pub. Date: Oct. 1, 2026

(54) NATURAL AQUEOUS FILM-FORMING COMPOSITION BASED ON A PAIR OF STARCHES

(71) Applicant: SWISS APPLIED COATING RESEARCH GMBH, Gentilino (CH)

(72) Inventor: Francesco Roversi, Gentilino (CH)

(73) Assignee: SWISS APPLIED COATING RESEARCH GMBH, Gentilino (CH)

(21) Appl. No.: 19/480,808

(22) PCT Filed: Apr. 30, 2024
(86) PCT No.: PCT/IB2024/054192

§ 371 Date: Nov. 1, 2025

(30) Foreign Application Priority Data
May 5, 2023 (IT) 102023000009042

(51) Int. Cl. A61K 9/28; A61K 9/48

(52) U.S. Cl. CPC A61K 9/286; A61K 9/2826; A61K 9/2893; A61K 9/4891

(57) ABSTRACT

The object of the invention relates to a water-based film-forming composition and to the respective film for the coating of oral solid forms and for rigid and soft capsules, comprising: acetylated pregelatinized starch containing amylose in an amount ≥50% by weight, and acetyl groups in an amount ranging from 0.5% to 2.5% by weight based on the weight of the starch; acetylated non-pregelatinized starch containing amylose in an amount ≥50% by weight, and acetyl groups in an amount ranging from 0.5% to 2.5% by weight based on the weight of the starch. This composition is characterized in that it is free of or does not require lubricants, in particular is talc-free.

### FIELD OF THE INVENTION

The field of the present invention relates to a film-forming composition based on a pair of acetylated starches, only one of which being pregelatinized, for the coating of pharmaceutical oral solid formulations, food supplements and/or for the coating of seeds.

### BACKGROUND ART

There are different types of starch depending on the source variety, for example corn, rice, potato and tapioca, and the different percentage of amylopectin and amylose. High-amylose starches (“HASs”) have at least 50% of this substance based on the total weight. They can either originate from particular plant hybrids or are prepared by enriching with amylose starches having a poor content of this component.

Amylose imparts consistency, heat stability, water resistance and adhesiveness properties, therefore high-amylose starches (hereinafter, HASs) exhibit undoubted advantages over other starches. Examples of HASs are those commercially available under the trademarks Eurylon™, Amylon™, Exylon™ and Nabond™.

HASs are used either as such or modified through various physical or chemical processes. One of the physical processes is pre-gelling, wherein the starch undergoes a heat treatment in the presence of water, thereby losing its granular form and becoming water-soluble at lower temperatures. Among the chemical processes, the esterification and the etherification, e.g. the acetylation, can be mentioned.

The esterification and etherification processes are used in several sectors, such as the food and pharmaceutical industries.

As far as the use in the food field is concerned, can be mentioned, by way of example: U.S. Pat. No. 3,751,268 (to American Maize-Products Company), disclosing the use of a non-modified and non-gelled HAS for the coating of foods to be fried in oil; U.S. Pat. No. 4,487,786 (to Lamb-Weston Inc.) disclosing a method for coating frozen foods with a mixture comprising a HAS, wheat flour and a food acid.

In the pharmaceutical field, HASs are used for several purposes.

EP 0 964 000 (to Roquette Frères) discloses a diluting and disintegrating composition containing a HAS, preferably Eurylon™, embedded in a pre-gelled starch matrix that can be used in order to produce solid forms, such as powders or tablets. The HAS is not acetylated.

U.S. Pat. No. 5,554,385 (to R.P. Scherer Corporation) refers to soft gelatine capsules, wherein a certain percentage of gelatin is replaced by a HAS. These capsules show a lower tendency to stick to each other, are more resistant and have a better appearance. The HAS is not acetylated.

U.S. Pat. No. 4,026,986 (to The Dow Chemical Company) refers instead to capsules that can be entirely made of a hydroxyalkylated HAS. These capsules show the advantage of being more uniform, more stable to water and high temperatures during the manufacturing process, and cheaper. The HAS is not acetylated.

GB 810,306 (to The Upjohn Company) claims an amylose diester, preferably an amylose acetate phthalate that can be used as coating for gastroresistant pharmaceutical preparations. However, this kind of coating has a disadvantage due to the permanence in the finished product of possible phthalic acid residues which should absolutely be removed through a quite complex process consisting of four re-suspensions in deionized water at room temperature, followed by a treatment with boiling water.

U.S. Pat. No. 4,738,724 (to Warner-Lambert Company) claims capsules for pharmaceutical use made of a mixture of starch and water. The manufacturing process of these capsules is extremely complex and it is not considered to be industrially advantageous. The method can also involve pregellation and derivation treatments with starch acetate.

The degree of acetylation is not specified. The method involves adding at least one lubricant, such as talc.

U.S. Pat. No. 5,281,276 (to National Starch and Chemical Investment Holding Corp.) describes a method for obtaining a starch which is resistant to amylase, i.e. the enzyme hydrolyzing starch in the intestine. The method involves gellating a starch having an amylose content greater than 40% and subsequently treating it with an enzyme capable of “de-branching” starch chains (“debranching enzyme”). The starch is not acetylated.

U.S. Pat. No. 5,224,989 describes coating compositions based on oxidized starch acetate having a degree of acetylation comprised between 0.5% and 2.4% for use in forming a protective coating on drug and food items. The amylose content is not specified. The compositions can contain fillers/diluents, such as talc or titanium oxide.

Die Pharmazie, 1993 (March), number 3, pp 224-225, illustrates the use of a non-pregelled acetylated starch.

U.S. Pat. No. 3,553,196 describes transparent, resistant, flexible and water-soluble films comprising acetylated starch having a high degree of acetylation, specifically between 0.21 and 0.31. The starch is not pre-gelled. In order to obtain acetylated starch granules that disintegrate and/or solubilize in water, however without degrading the acetylated starch, a continuous treatment by an “injection cooker” equipment (“continuous steam injection cooker”) at a relatively high temperature (about 177° C.) is provided.

U.S. Pat. No. 5,514,790 describes the preparation of an acetylated starch. The process involves a step of activating the hydroxyl groups of the starch by using soda, followed by precipitation, filtration and drying. Once activated, the starch can be modified or derivatized with acetal groups. These acetylated starches dissolve in non-aqueous solvents, such as acetone, chloroform.

The coating of pharmaceutical, food, dietary and cosmetic forms for humans and animals and the coating of seeds in the agriculture sector have several functions. For example, in the pharmaceutical industry, there exists the need to cover the unpleasant taste of some active ingredients or compositions; the need to protect the formulation from ambient humidity, which can cause degradation phenomena; or, further, the need to obtain a gastroresistance effect, so that the drug or the active agent reaches intact the intestine in order to be absorbed. Similarly, the same needs exist in the food industry and in the agriculture sector.

For example, this issue is particularly relevant in the food field since, for legislative reasons, it is not possible to use the acrylic and methacrylic polymers, such as Eudragit™, commonly used in the pharmaceutical field. The only material that can serve the aforementioned purposes and can be legally used is shellac, which, however, shows issues related to batch, weight and structure uniformity, besides requiring solvents different from water for its processing and application.

On the other hand, coatings based on 25% shellac aqueous solutions, such as those under the tradename AquaGold® by SSB, are commercially known for serving the same purpose as Eudragit.

Beyond these specific issues, the need for non-toxic materials which are well tolerated by organisms still exists in both sectors. For example, some users find it difficult to digest gelatin-based coatings; in addition, more and more people require and prefer pharmaceutical and food/dietary preparations containing as much natural products or products of natural origin as possible.

EP1296658 discloses the use of an acetylated pre-gelatinized starch that is soluble in water at 60-80° C., containing at least 50% by weight of amylose and a percentage of acetyl groups between 0.5% and 2.5% by weight, for use in the manufacturing of capsules, for the coating of solid formulations in pharmacological, cosmetic, food and dietary fields, both for humans and for animals, and for the coating of seeds. Formulations having the coating based on the aforementioned acetylated pregelatinized starch require the presence of at least one lubricant, such as selected from: PVP, glycerol monostearate, colloidal silica, kaolin, titanium oxide, magnesium stearate, alginates, talc and mixtures thereof, preferably comprising talc.

Lubricants (or anti-adherents) are well-known excipients reducing friction in solid formulations; some lubricants, such as talc, can also act as diluents. These substances can be potentially carcinogenic per se and/or are often found in the form of small-sized particles, e.g., nanoparticles, that could be toxic and/or carcinogenic upon absorption, such as during the processing or manufacturing steps of a pharmaceutical formulation. For example, in some countries, such as the United States, Canada or Japan, the presence of talc is not appreciated or allowed from a regulatory standpoint.

There exists the need to obtain simpler film-forming compositions or film-forming agents requiring fewer natural and/or plant-based components/ingredients, and possibly at a lower cost. It is also required that such film-forming compositions are able to achieve at least the same level of technological efficiency as the known state-of-the-art film-forming compositions or agents, i.e., such as those including lubricants and/or diluents, in order to obtain films provided with appropriate mechanical strength, for manufacturing capsules and coatings for solid formulations used in the pharmacological, cosmetic, food and dietary fields, as well as in the agriculture sector. Finally, it is further important that such film-forming compositions also show a certain functionality, such as gastroresistance.

### SUMMARY OF THE INVENTION

A first object of the present invention is a film-forming aqueous composition comprising:
a) acetylated pregelatinized starch containing amylose in an amount ≥50% by weight, and acetyl groups in an amount ranging from 0.5% to 2.5% by weight based on the weight of the starch,
b) acetylated non-pregelatinized starch containing amylose in an amount ≥50% by weight, and acetyl groups in an amount ranging from 0.5% to 2.5% by weight based on the weight of the starch.

A further object of the invention is the use of the film-forming aqueous composition for the coating of delayed-release solid formulations, and the use of the film as coating for solid formulations, such as tablets, powders or granules, for the coating of seeds, or soft and/or hard or rigid capsules.

The film-forming aqueous composition or the film-forming agent of the invention proves to be advantageous since:
it contains two different forms of starch, both acetylated, only one of which being pregelatinized, which contribute to make the film-forming composition capable of functioning in technological terms, easily processable, provided with mechanical strength and gastroresistance, in the absence of lubricating agents;
there is no need to modify the industrial facilities usually employed for preparing and obtaining the film on the desired substrate;
it is natural, which means that, as film-forming agents, it contains starches, i.e. natural-type substances;
it is versatile in that it can be employed for the filming of different solid formulations, both in the chemical-pharmaceutical and agricultural-food fields.

### DETAILED DESCRIPTION OF THE INVENTION

Hereinafter, the Applicant will describe the different objects of the invention in further detail.

For the purposes of the present invention, the definition “comprising” also includes components which are not explicitly listed after this definition. Instead, the definitions “consisting in” or “consisting of” exclude the presence of further components which are not explicitly mentioned after this definition.

Film-Forming Aqueous Composition and Film Obtained from Said Film-Forming Aqueous Composition

The film-forming aqueous composition (or aqueous composition or film-forming composition) of the invention comprises
a) acetylated pregelatinized starch containing
amylose in an amount ≥50% by weight, preferably ≥50% and ≤98% by weight, preferably ≥60% and ≤98% by weight, preferably ≥70% and ≤98% by weight, preferably ≥80% and ≤98% by weight, preferably ≥90% and ≤98% by weight, preferably ≥95% and ≤98% by weight, and
acetyl groups in an amount ranging from 0.5% to 2.5% by weight, preferably ranging from 1% to 2.3% by weight, preferably from 1.5% to 2% by weight, preferably from 1.4% to 1.7% by weight, preferably of 1.4% by weight, based on the weight of the starch,
b) acetylated non-pregelatinized starch containing
amylose in an amount ≥50% by weight, preferably ≥50% and ≤98% by weight, preferably ≥60% and ≤98% by weight, preferably ≥70% and ≤98% by weight, preferably ≥80% and ≤98% by weight, preferably ≥90% and ≤98% by weight, preferably ≥95% and ≤98% by weight, and
acetyl groups in an amount ranging from 0.5% to 2.5% by weight, preferably from 1% to 2.3% by weight, preferably from 1.5% to 2% by weight, preferably from 1.4% to 1.7% by weight, preferably of 1.4% by weight, based on the weight of the starch.

Preferably, the acetylated pregelatinized starch and the acetylated non-pregelatinized starch are “high-amylose” starches, which means that they preferably contain amylose in an amount >70%, preferably ≥75% by weight, preferably between 70% and 75% by weight, preferably of 75% by weight, based on the total weight of the starch.

As for the film-forming aqueous composition, the weight ratio of the pregelatinized acetylated starch a) to the non-pregelatinized acetylated starch b)—pregelatinized acetylated starch a)/non-pregelatinized acetylated starch b)—is preferably comprised between 2:1 and 10:1, preferably between 2:1 and 9:1, preferably between 3:1 and 9:1, preferably between 4:1 and 9:1, preferably between 4.5:1 and 9:1, preferably between 5:1 and 9:1, preferably between 6:1 and 7:1, preferably of 4.5:1, 5:1 or 9:1.

As for the film, the weight ratio of the pregelatinized acetylated starch a) to the non-pregelatinized acetylated starch b)—i.e., pregelatinized acetylated starch a)/non-pregelatinized acetylated starch b)—is preferably comprised between 2:1 and 10:1, preferably between 2:1 and 9:1, preferably between 3:1 and 9:1, preferably between 4:1 and 9:1, preferably between 4.5:1 and 9:1, preferably between 5:1 and 9:1, preferably between 7:1 and 9:1, preferably between 8:1 and 9:1, preferably of 9:1.

The pregelatinized starch a) and the non-pregelatinized starch b) are both acetylated, and they are preferably obtained by an acetylation method known to a person skilled in the art. An acetylation method is described, for example, in Starch, Whistler, Bemiller and Pascal eds., Academic Press, (1984), page 332 and ff. The acetylation is preferably carried out with acetic anhydride, thereby obtaining a percentage of acetyl groups greater than 0.5% by weight, but lower than 2.5% by weight.

Pregelled or pregelatinized starch means a starch that has been subjected to a pregellation or pregelatinization process. Non-pregelled or non-pregelatinized starch means a starch that has not been subjected to a pregellation process.

Pregelatinization process (or baking) means a method known to a person skilled in the art, wherein the starch, be it acetylated or not, is dispersed in an aqueous solution which is then poured into a rotating drum heated at a temperature preferably ranging between 100° C. and 130° C., operating at pressures between 2 and 3 bars; thereby, upon contact with the roll surface, the starch dispersion “explodes” resulting in the evaporation of water, preferably within a time ranging between 30 seconds and 3 minutes. A gel layer containing the starch is thus deposited onto the rotating drum surface. This gel solidifies and is removed by scraping. The thereby obtained starch gel preferably has a humidity percentage varying from 5% to 10% by weight based on the total weight of the pregelatinized starch.

For the purposes of the present invention, “film obtained from the film-forming aqueous composition” means a film or a dry composition obtained by removing water included within the film-forming aqueous composition. The film preferably contains a water content <5% by weight, preferably <3%, preferably <2% by weight, preferably <1%, preferably <0.5% by weight, preferably <0.1% by weight, preferably <0.05%, preferably <0.01% by weight, based on the total weight of the film; preferably, the film does not contain water other than that which can be inherently contained in the components included in the film.

Preferably, in the film-forming aqueous composition of the invention, the acetylated pregelatinized starch is in an amount ranging from 5% to 25% by weight, preferably from 7% to 23% by weight, preferably from 10% to 20% by weight, preferably from 12% to 18% by weight, preferably from 12% to 17% by weight, preferably from 13% to 15% by weight, preferably of 13.5% or 15% by weight, based on the total weight of the composition, when it contains water.

Preferably, the acetylated pregelatinized starch in the film obtained from the aqueous composition is in an amount ranging from 60% to 80% by weight, preferably from 65% to 80% by weight, preferably from 66% to 80% by weight, preferably from 67% to 78% by weight, preferably from 67% to 76% by weight, preferably of 67.6% or 75% by weight, based on the total weight of the film.

Preferably, the acetylated non-pregelatinized starch in the film-forming aqueous composition is in an amount ranging from 0.5% to 3.5% by weight, preferably from 0.7% to 3.5% by weight, preferably from 1% to 3% by weight, preferably from 1.5% to 3% by weight, preferably from 1.5% to 2.7% by weight, preferably from 1.7% to 2.7% by weight, preferably of 1.7% or 2.7% by weight, based on the total weight of the composition.

Preferably, the acetylated non-pregelatinized starch in the final film obtained from the film-forming aqueous composition according to the present invention is in an amount ranging from 6% to 15% by weight, preferably from 7% to 15% by weight, preferably from 7.5% to 14% by weight, preferably from 8% to 14% by weight, preferably from 8.3% to 13.5% by weight, preferably of 8.3% or 13.5% by weight, based on the total weight of the film.

Preferably, the film-forming aqueous composition contains a total starch content (acetylated pregelatinized starch and acetylated non-pregelatinized starch) comprised between 13% and 18% by weight, preferably between 13% and 17% by weight, preferably between 14% and 17% by weight, preferably between 14% and 16.6% by weight, based on the total weight of the composition.

An acetylated pregelatinized starch is, for example, the commercial product AMPRAC-01; while, as non-pregelatinized starch, the commercial product AMPRAC-03 can be preferably employed.

Note that the acetylated pregelatinized starch and the acetylated non-pregelatinized starch are in the above-mentioned amounts in order to ensure the film-forming composition of the invention mechanical strength and/or gastroresistance.

Preferably, the pregelatinized or non-pregelatinized acetylated starch can be a high-amylose starch which can be found in nature originating from various sources, such as: corn, potato, rice, wheat, sorghum, tapioca. Preferably, the starch is corn starch.

Note that, alternatively, the high-amylose pregelatinized or non-pregelatinized acetylated starch can be preferably obtained by enriching with amylose any one of the commercially available starches.

The film-forming aqueous composition preferably does not contain preserving agents and/or synthetic polymers and/or preservatives.

The film-forming aqueous composition has a shelf-life preferably ≤2 days, preferably of one or two days, when maintained at a temperature between 40° C. and 55° C., preferably of about 50° C.

The film-forming composition preferably comprises at least one plasticizer.

The film-forming composition comprises a plasticizer or plastifier selected from the group consisting of: sorbitol, glycerol, triethyl citrate, polysorbate, carnauba wax, PEG and mixtures thereof; preferably, the plasticizer is sorbitol and/or glycerol.

Preferably, the plasticizer is in an amount ranging from 1% to 5% by weight, preferably from 1.5% to 4.5% by weight, preferably from 2% to 4% by weight, preferably from 2.5% to 3.5% by weight, preferably from 3% to 3.5% by weight, preferably of 3.4% or 3% by weight, based on the total weight of the aqueous composition.

Preferably, in the film obtained with the aqueous composition of the invention, the plasticizer is in an amount ranging from 13% to 20% by weight, preferably from 13% to 19% by weight, preferably from 13% to 18% by weight, preferably from 14% to 18% by weight, preferably from 14% to 17% by weight, preferably of 14.7% or 17% by weight, based on the total weight of the film.

Note that, preferably, the polymer-based film-forming compositions, as in the present invention, require at least one plasticizer in order to ensure elasticity, and mechanical strength and stability over time, of the film obtained from such an aqueous composition. For example, for the purposes of the invention, by employing at least one plasticizer, there is no need to modify the spraying facilities for coating processes of a solid formulation.

In the starting aqueous composition, water is preferably contained in an amount ranging from 60% to 90% by weight, preferably from 65% to 87% by weight, preferably from 70% to 85% by weight, preferably from 75% to 80% by weight, preferably from 77% to 80% by weight, preferably of 80% by weight, based on the total weight of the composition.

Preferably, the subject aqueous composition comprises at least one agent capable of imparting gastroresistance (also referred to as a coating agent or ingredient) to the film for the coating of solid formulations or for soft and/or hard capsules.

Preferably, the film contains at least one coating agent or ingredient, preferably a coating ingredient, capable of imparting gastroresistance to the film. Preferably, the film obtained from the film-forming aqueous composition is gastroresistant.

In case the film obtained from the aqueous composition constituting the object of the present invention is also gastroresistant, the aqueous composition constituting the object of the present invention comprises an agent capable of imparting gastroresistance to the film for the coating of solid formulations or for soft capsules.

Preferably, this agent can be selected from those synthetically obtained or even better from natural ones or from those of natural origin, such as lac or shellac.

Preferably, the at least one coating ingredient is in an amount ranging from 0.3% to 1.5% by weight, preferably from 0.5% to 1% by weight, preferably from 0.5% to 0.9% by weight, preferably from 0.7% to 0.9% by weight, preferably of 0.81% by weight, based on the total weight of the film-forming aqueous composition.

Preferably, the at least one coating ingredient is in an amount ranging from 2% to 5% by weight, preferably from 3% to 5% by weight, preferably from 3.5% to 4% by weight, preferably of 4% by weight, based on the total weight of the film obtained from the film-forming composition constituting the object of the invention.

Preferably, the at least one pre-coating ingredient is in an amount <7% by weight, preferably ≤6% by weight, preferably ≤5% by weight, preferably between 0.5% and 5%, preferably between 1% and 5%, preferably of 5% by weight, based on the total weight of starches in the film-forming composition of the invention.

Note that the at least one pre-coating ingredient is preferably employed in lower percentages compared to those normally required for the state-of-the-art compositions;

in the latter case, the coating ingredients are in an amount preferably >7% by weight, preferably >10% by weight, based on the total weight of starches in the state-of-the-art film-forming compositions.

Note that the film-forming aqueous composition is preferably substantially or exclusively natural.

Natural means that the composition comprises substantially or exclusively natural ingredients or ingredients of natural origin.

Substantially natural means that the film-forming aqueous composition preferably comprises a concentration of natural ingredients or ingredients of natural origin ≥80%, preferably ≥85% by weight, ≥90% by weight, ≥95% by weight, preferably between 95% and 99% by weight, preferably between 96% and 99% by weight, preferably between 97% and 99% by weight, preferably between 98% and 99% by weight, based on the total weight of the composition.

Exclusively natural means that the film-forming aqueous composition comprises only natural ingredients or ingredients of natural origin, preferably in a concentration of 100% by weight based on the total weight of the composition.

For the purposes of the invention, and in accordance with ISO 16128 standard, “natural ingredient” or “ingredient of natural origin” means an ingredient obtained exclusively from plants, animals, microorganisms or minerals, by: physical processes (e.g., grinding, drying, distillation); fermentation reactions, as occurring in nature and resulting in naturally occurring molecules; other preparation processes, including traditional ones (e.g., solvent extraction) without intentional chemical modifications. Ingredients obtained from fossil fuels are excluded from the above definition.

For the purposes of the invention, and in accordance with ISO 16128 standard, “derived natural ingredient” means an ingredient obtained by chemical and/or biological processes with the intent to chemically modify it (e.g., the enzymatic and microbiological processing can originate natural ingredients or derived natural ingredients in which an intentional chemical modification occurs). The degree of natural origin is usually quantified based on the molecular weight or the renewable carbon content of an ingredient.

### Lubricants

Preferably, the film-forming composition does not comprise lubricants.

For example, the film-forming composition does not contain: polyvinylpyrrolidone, glycerol monostearate, colloidal silica, kaolin, titanium dioxide, magnesium stearate, alginates, talc and mixtures thereof. The film-forming aqueous composition or the film obtained from the aforementioned composition preferably do not contain talc, which instead is provided in large amounts in the Patent EP1296658.

Note that preferably the characteristics of each ingredient/component indicated above are as described in the previous paragraphs.

According to the first, second and third preferred embodiments, the film-forming composition can contain dyes.

According to another preferred embodiment, the film-forming composition does not contain ingredients selected from the group consisting of: agents modulating the ability of the coating to dissolve within certain amounts of time or at certain pH values; agents imparting gastroresistance selected from the group consisting of: acacia, cellulose; synthetic polymers (acrylic polymers); and mixtures thereof. These ingredients can generally be contained in amounts between 10 and 70% by weight relative to the weight of the starch.

### Preparation Process of the Film-Forming Aqueous Composition

The preparation process of the film-forming aqueous composition involves the following steps:
i) heating the water, preferably by means of suitable equipment;
ii) once a temperature between 40° C. and 65° C., preferably between 50° C. and 60° C., has been reached, adding the acetylated pregelatinized starch and the acetylated non-pregelatinized starch, preferably under appropriate stirring, to obtain an aqueous solution;
iii) then, bringing the aqueous solution to a temperature between 75° C. and 85° C., preferably between 80° C. and 85° C., under stirring, preferably letting it stir for a time period between 20 and 40 minutes, preferably of 30 minutes;
iv) optionally, adding any other suitable excipients and/or diluents.

Note that the temperature affects the preparation time of the composition, the baking of the starch (it is effective only when 100% baked) and the viscosity.

With this process, there is no need to modify the industrial facilities usually employed for preparing and obtaining the film on the desired substrate.

### Use of the Film-Forming Composition

A further object of the invention is the use of the film-forming composition for the coating of solid formulations and/or for the coating of seeds.

Solid formulations mean those selected from the group consisting of: powders, granulates, microgranules, pellets, tablets, capsules, preferably for humans and/or animals. Preferably, they can be solid formulations used in the pharmacological, cosmetic, food, dietary, agricultural, veterinary fields.

With the present film-forming composition it is possible to obtain coatings selected from the group consisting of: functional coatings, such as gastroresistance, colon delivery, delayed-release; moisture and/or light protective coatings; for masking unpleasant odors and tastes; controlled or delayed-release coatings, and mixtures thereof.

The film-forming aqueous composition, preferably when the composition comprises at least one pre-coating ingredient, can be used for the coating of delayed-release solid formulations.

For the purposes of the invention, the film of the invention is employed for the filming of solid formulations; in this sense, the film of the invention preferably constitutes the outermost coating layer of the solid formulation. Preferably, still for the filming of solid formulations, it is possible to add an inner layer (i.e., below the outermost coating layer of the film of the invention), said inner layer being very thin and consisting of the so-called pre-coating ingredient.

Pre-coating ingredient means an ingredient that can be employed for forming a very thin inner layer that can be preferably inserted below the outermost coating layer comprising or consisting of the film of the invention.

Note that preferably, for the purposes of the invention, the delayed-release and/or gastroresistance effect is essentially imparted by the film of the invention; any other pre-coating ingredients that may be present assist the action of the film of the invention, however their presence is not essential for the purposes of the above-mentioned effect.

Preferably, the film of the invention can be used for the coating of solid formulations, for the coating of seeds, for the coating of soft capsules; and/or for the coating of delayed-release solid formulations.

The filming and/or coating process of solid formulations is known to the person skilled in the art.

For example, solid formulations, such as tablets and/or capsules, can be coated by spraying or filming in a coating pan. Furthermore, for example, for manufacturing soft and/or hard capsules using the film-forming composition of the invention, the methods to be followed are those commonly known in the field (e.g., U.S. Pat. No. 2,299,039).

On the other hand, preferably, pellets and microgranules are usually coated by a filming technique at least selected from: in a fluidized bed, spray drying, nebulization, ultrasonic atomization, rotogranulation or spheronization.

Preferably, the coating procedure involves suspending in water the high-amylose acetylated and pregelatinized starch together with the starch, possibly with suitable excipients and/or diluents, at a temperature between 50° C. and 60° C. A concentration of solids in aqueous suspension between 10% and 25% by weight, preferably of 20% by weight, based on the weight of the total aqueous suspension, is obtained.

Depending on the type of coating to be obtained, a different amount of starch per unit area to be coated is used in the covering processes.

### EXAMPLES

Below, the Applicant will provide some examples for merely illustrative, but non-limiting purposes.

### Example 1—Film-Forming Composition of the Invention for Tablets and Soft Gelatin Capsules

| | | Total |
|---|---|---|
| | | amount or |
| | Dry | including |
| Ingredients | amount (g) | water (g) |
| Pregelatinized acetylated | 373.50 | 373.50 |
| starch (90%) or | | |
| AMPRAC-01 | | |
| Non-pregelatinized | 41.50 | 41.50 |
| acetylated starch (10%) | | |
| or AMPRAC-03 | | |
| Glycerol | 83.00 | 83.00 |
| Water | / | 1992.00 |
| Total | 498.00 | 2490.00 |

### Preparation Method:

The water needed for the formulation is heated by a dedicated equipment; when 50°-60° C. is reached, the two starches are added as a mixture under appropriate stirring. The solution is brought to 80° C. and let to stir for at least another 30 minutes; thereafter, the plasticizer and any necessary excipients and/or dyes are added.

### Example 2—Gastroresistant Film-Forming Composition of the Invention

| | | Total |
|---|---|---|
| | | amount or |
| | Dry | including |
| Ingredients | amount (g) | water (g) |
| Pregelatinized acetylated | 745.00 | 745.00 |
| starch (90%) or | | |
| AMPRAC-01 | | |
| Non-pregelatinized | 149.00 | 149.00 |
| acetylated starch (10%) | | |
| or AMPRAC-03 | | |
| Glycerol | 162.50 | 162.50 |
| Shellac | 45.00 | 45.00 |
| Water | / | 4406.00 |
| Total | 1101.50 | 5507.50 |

Note that such a composition can be employed for preparing tablets and soft or rigid capsules, preferably delayed-release ones.

### Preparation Method:

The water needed for the formulation is heated by a dedicated equipment; when 50°-60° C. is reached, the two starches are added as a mixture under appropriate stirring. The solution is brought to 80° C. and let to stir for at least another 30 minutes; thereafter, the plasticizer and any necessary excipients and/or dyes are added.

### Example 3—Disgregation Test

Disgregation test according to current Pharmacopoeiae (Ph. Eur., USP/NF, FOOD CHEMICALS CODEX, JEFCA specifications, US 21 CFR, JPE) for delayed-release solid forms.

For this test, the composition according Example 2 of the invention was used.

The Applicant noticed that, in an acidic environment (pH 1.2), no disgregation or dissolution or substance release is observed for 1-2 hours, according to the required Pharmacopoeia.

In contrast, in the enteric environment (pH 6.8), a complete disgregation of the nucleus after 1 hour residence time is observed. This indicates that the composition of the invention shows gastroresistant ability and is consistent with current Pharmacopoeiae.

1. A film-forming aqueous composition, comprising:
a) acetylated pregelatinized starch containing amylose in an amount ≥50% by weight, and acetyl groups in an amount ranging from 0.5% to 2.5% by weight based on the weight of the starch,
b) acetylated non-pregelatinized starch containing amylose in an amount ≥50% by weight, and acetyl groups in an amount ranging from 0.5% to 2.5% by weight based on the weight of the starch,
wherein the composition does not contain lubricants.

2. The film-forming aqueous composition according to claim 1, wherein the acetylated pregelatinized starch and the acetylated non-pregelatinized starch contain amylose in an amount >70%.

3. The film-forming aqueous composition according to claim 1, wherein the weight ratio of acetylated pregelatinized starch a) to acetylated non-pregelatinized starch b) ranges from 2:1 to 10:1.

4. The film-forming aqueous composition according to claim 1, wherein the acetylated pregelatinized starch a) is in an amount ranging from 5% to 25% by weight on the total weight of the composition.

5. The film-forming aqueous composition according to claim 1, wherein the acetylated non-pregelatinized starch b) is in an amount ranging from 0.5% to 3.5% by weight based on the total weight of the composition.

6. The film-forming aqueous composition according to claim 1, wherein the starch is corn starch.

7. The film-forming aqueous composition according to claim 1, further comprising a plasticizer.

8. The film-forming aqueous composition according to claim 7, wherein the plasticizer is selected from the group consisting of: sorbitol, glycerol, triethyl citrate, polysorbate, carnauba was, PEG and mixtures thereof.

9. The film-forming aqueous composition according to claim 7, wherein the plasticizer is in an amount ranging from 1% to 5% by weight based on the total weight of the composition.

10. The film-forming aqueous composition according to claim 1, comprising at least one pre-coating agent suitable for gastroresistance.

11. (canceled)

12. A film obtained from the aqueous composition according to claim 1.

13. Method for coating solid formulations, seeds, hard and soft capsules, orosoluble films, pellets, micro-tablets, granulates, powders for the pharmaceutical, food supplement, veterinary industries, and for agriculture sector, said method comprising:
applying said aqueous film-forming composition according to claim 1 onto said solid formulations, seeds, hard and soft capsules, orosoluble films, pellets, micro-tablets, granulates, powders and
drying said aqueous film forming composition applied onto said solid formulations, seeds, hard and soft capsules, orosoluble films, pellets, micro-tablets, granulates, powders for the pharmaceutical, food supplement, veterinary industries, and for agriculture sector.

14. The film forming aqueous composition according to claim 1 wherein in the weight ratio of acetylated pregelatinized starch a) to acetylated non-pregelatinized starch b) ranges from 6:1 to 7:1.

15. The film forming composition according to claim 1 the weight ratio of acetylated pregelatinized starch a) to acetylated non-pregelatinized starch b) is selected from 4.5:1 or 5:1.

16. The film-forming aqueous composition according to claim 7, wherein the plasticizer is selected from glycerol and/or sorbitol.

17. The film-forming aqueous composition according to claim 10, wherein said at least one pre-coating agent is shellac.
